(19) United States
(12) Patent Application Publication
(10) Pub. No.: US 2026/0328745 A1
(43) Pub. Date: Oct. 22, 2026

(54) METHOD AND APPARATUS FOR CONTINUOUS PLANT HEALTH MONITORING

(71) Applicants: Board of Regents, The University of Texas System, Austin, TX (US); The Regents of the University of Colorado, Denver, CO (US)

(72) Inventors: VP NGUYEN, Austin, TX (US); Tam VU, Boulder, CO (US); Tuan DANG, Austin, TX (US); Tien PHAM, Austin, TX (US); Nhat PHAM, Boulder, CO (US)

(21) Appl. No.: 19/115,050

(22) PCT Filed: Oct. 3, 2023
(86) PCT No.: PCT/US2023/075825

§ 371 Date: Mar. 25, 2025

Related U.S. Application Data
(60) Provisional application No. 63/412,753, filed on Oct. 3, 2022.

(51) Int. Cl. G01N 33/00; G01N 27/02

(52) U.S. Cl. CPC G01N 33/0098; G01N 27/026

(57) ABSTRACT

According to some embodiments, a system for monitoring nutrient levels in the body of a tree is provided, the system comprising: a sensing component comprising a sensor implanted into the tissue of the tree to generate a dataset that corresponds to information about the tree, a communication component to transmit data, the data comprising at least the generated dataset, and a base station to receive the data.

### CROSS-REFERENCE TO RELATED APPLICATIONS

This application claims priority pursuant to Article 8 of the Patent Cooperation Treaty to U.S. Provisional Patent Application Ser. No. 63/412,753 filed Oct. 3, 2022, which is incorporated herein by reference in its entirety.

### FIELD

The technology described herein generally relates to biocompatible sensing systems, more specifically to low power and/or battery-free biocompatible sensing systems for the continuous monitoring of plants.

### BACKGROUND

Global agriculture will need to produce more food in the next 50 years than in the previous ten thousand years in order to feed the growing population. In modern agriculture, a few crops known to have high yields are selected to grow intensively. But growing just a few varieties of trees makes our food supply vulnerable to pests, diseases, climate changes, leading to overuse of fertilizers and chemicals also negatively affects soil health, creating a vicious cycle and making our farmlands less productive and the food we grow less nutritious. In the US, more than 1,700 trillion BTU (approximately $17 billion) of energy was consumed by agriculture every year, and nearly 30% of this was for the production of fertilizer; yet, nitrogen fertilizer has a use efficiency of 33% globally. Understanding how trees on the farm are growing and interacting with their environment will help reduce the use of fertilizers, chemicals, and precious resources like water, and design novel and sophisticated growing techniques like intercropping and cover cropping to restore soil fertility and increase productivity.

Advanced soil sensors, camera-based sensors, and sap sample analyses have been proposed or utilized to monitor and derive water and nutrient uptake behaviors of the trees. However, these sensors are not able to provide reliable insights since the data are not derived from the body of the tree. Moreover, as these sensors are generally required to be buried at 40 cm below the ground, the deployment cost is high, and the soil-to-air wireless communication channel is unreliable. Second, camera-based solutions cannot provide internal signals of the tree and additionally, perform poorly under low-light, rain, and dusty conditions. Last, sap samples extracted from the tree's xylem in a farm setting can be analyzed offline later in the lab to infer the water and nutrient contained inside the tree, but these techniques are costly, complex, time-consuming, and labor intensive. In addition, as the measurements are not obtained from a living tree or plant, the real-time impact of the environments on the tree or plant are unclear.

Related techniques, such as nutrient monitoring techniques or agricultural sensing, are widely used but are also accompanied by multiple deficiencies. For instance, in a nutrient monitoring technique, measuring ion quantities directly is the most accurate way to monitor nutrients inside trees or plants. Ion-selective electrodes are widely used in commercialized products and in literature to measure, for instance, temperature, pH, sodium chloride, ammonium phosphatides, electrical conductivity, dissolved oxygen from soil or the hydroponic solution supplied to trees. However, these systems require humans to operate and are highly expensive, making it challenging to be used on an individual tree. Other approaches such as employing ultra-violet spectral absorption and wet chemical colorimetric reaction to measure individual ions such as ammonium, nitrate, and phosphate in the hydroponic solution have been used and have shown high accuracy and fast response time. Nevertheless, these systems are costly (generally over $10 k). Thus, it is impossible to scale these systems to monitor the nutrient characteristics of an individual tree on a large scale. Another approach is to infer the nutrient level inside a tree based on the leaves' color. However, this approach is inaccurate due to the lack of information retrieving directly from the inside of the trees and is inapplicable during the defoliation period (when the tree has no leaves).

In agricultural sensing platforms, automatic irrigation systems based on soil moisture, and IoT-based systems based on sensors attached on trees are widely adopted in both literature and practice to improve crop yield and water efficiency effectively. However, they have several limitations, such as (1) the dependency on a power supply station and (2) the use of invasive sensors that can harm trees. The most common way to monitor nutrient deficiencies is based on the morphology and color of the leaves, which is impossible during the dormant stage. Previously implemented sensors are not suitable for large trees with large barks. A self-powered sensor for agriculture use was also developed however, this sensor is capable of Bluetooth communication only, which is impractical in farm settings since Bluetooth has a limited range of communications. Lastly, there is no previous work that integrates intermittent computing into agriculture-based applications. Therefore, existing or conventional systems cannot monitor trees' condition while being accurate, biocompatible, battery-free, and extendable to a large scale, and provide longer-range communications.

Accordingly, the technology described herein provides improvements over conventional sensing and monitoring techniques and applications through a wind-powered, low maintenance, battery-free, biocompatible, implantable, tree wearable, and intelligent sensing system to continuously capture the signals inside the body of a living tree or plant to infer its water and nutrient levels.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

At a high level, embodiments of the technology described herein are generally directed towards a wind-powered, low maintenance, battery-free, biocompatible, implantable, tree-wearable, and intelligent sensing system (referred to herein as a biocompatible sensing and/or monitoring system, or otherwise “IoTree”) that can continuously capture signals inside the body of a living tree to generate and/or infer its water and nutrient levels. In some other embodiments, a biocompatible sensing and/or monitoring system can capture one or more signals inside the bodies of a plurality of trees and/or plants to either in real-time or subsequently, based on a collected or generated data set, infer water and/or nutrient levels and or changes.

According to some embodiments, a system for monitoring nutrient levels in the body of a tree are provided, the system comprising: a sensing component comprising a sensor implanted into the tissue of the tree to generate a dataset that corresponds to information about the tree, a communication component to transmit data, the data comprising at least the generated dataset, and a base station to receive the data.

According to some even further embodiments, a computer-implemented method for monitoring nutrient levels in the body of a tree is provided, the method comprising: implanting one or more sensors into a tree body, capturing one or more signals from the tree to generate a dataset corresponding to the tree, determining an impedance and/or impedance profile for the tree, and transmitting the at least one of the dataset, the impedance and/or impedance profile to a base station.

According to some even further embodiments, a system is provided having a computer storage medium storing computer-useable instructions that, when used by one or more computing devices, cause the one or more computing devices to perform operations comprising: capturing one or more signals from the tree to generate a dataset corresponding to the tree, determining an impedance and/or impedance profile for the tree, and transmitting the at least one of the dataset, the impedance and/or impedance profile to a base station.

Additional objects, advantages, and novel features of the technology will be set forth in part in the description which follows, and in part will become apparent to those skilled in the art upon examination of the following, or can be learned by practice of the invention.

### BRIEF DESCRIPTION OF THE DRAWINGS

Aspects of the technology described herein can be better understood with reference to the following drawings. The components in the drawings are not necessarily to scale, with emphasis instead being placed upon clearly illustrating the principles of the disclosure. Aspects of the technology presented herein are described in detail below with reference to the accompanying drawing figures, wherein:

FIG. 1 shows a schematic illustrating a biocompatible sensing and monitoring system, in accordance with some aspects of the technology described herein;

FIG. 2 illustrates a biocompatible sensing and monitoring system and components, in accordance with some aspects of the technology described herein;

FIG. 3 illustrates a schematic of an example sensor and working aspects of an impedance sensing unit, in accordance with some aspects of the technology described herein;

FIG. 4 illustrates an impedance profile measured by a VSP potentiostat, in accordance with some aspects of the technology described herein;

FIG. 5A illustrates a harvesting circuit diagram, in accordance with some aspects of the technology described herein;

FIG. 5B illustrates aspects of voltage and current of energy harvested at different wind speeds, in accordance with some aspects of the technology described herein;

FIG. 6 illustrates wind energy distribution at different distances as compared to solar energy, in accordance with some aspects of the technology described herein;

FIG. 7 illustrates adaptive block sizing for achieving optimal system configuration to minimize wasted energy, in accordance with some aspects of the technology described herein;

FIG. 8 illustrates aspects of a non-volatile buffer manager for use in a system, in accordance with some aspects of the technology described herein;

FIG. 9 illustrates various aspects of a biocompatible sensing and monitoring system, in accordance with some aspects of the technology described herein;

FIG. 10 illustrates sweeping power frequency power consumption of a biocompatible sensing and monitoring system, in accordance with some aspects of the technology described herein;

FIG. 11 illustrates experimental data with nitrogen and potassium showing impedance profiles for different levels of nutrients, in accordance with some aspects of the technology described herein;

FIG. 12A illustrates sensing accuracy of a biocompatible sensing and monitoring system in accordance with some aspects of the technology described herein;

FIG. 12B illustrates sensing accuracy of a biocompatible sensing and monitoring system in accordance with some aspects of the technology described herein;

FIG. 13 illustrates water and nutrient sensing on living trees by a biocompatible sensing and monitoring system, in accordance with some aspects of the technology described herein;

FIG. 14 illustrates data transmission measurements of a deployed biocompatible sensing and monitoring system, in accordance with some aspects of the technology described herein;

FIG. 15 illustrates example sensing data in a deployed biocompatible sensing and monitoring system under different wind conditions, in accordance with some aspects of the technology described herein;

FIG. 16A illustrates energy considerations associated with a biocompatible sensing and monitoring system, in accordance with some aspects of the technology described herein;

FIG. 16B illustrates memory usage considerations associated with a biocompatible sensing and monitoring system, in accordance with some aspects of the technology described herein;

FIG. 17 illustrates an example deployment of a biocompatible sensing and monitoring system, in accordance with some aspects of the technology described herein;

FIG. 18 illustrates long-range communication success at various distances for a biocompatible sensing and monitoring system, in accordance with some aspects of the technology described herein;

FIG. 19 illustrates example aspects of algorithm(s) and/or pseudo-code that can be used in an implementation of a biocompatible sensing and monitoring system, in accordance with some aspects of the technology described herein; and

FIG. 20 is a block diagram of an example computing environment and/or device architecture in which some implementations of the present technology may be employed.

### DETAILED DESCRIPTION

The subject matter of aspects of the present disclosure is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” can be used herein to connote different elements of methods employed, the terms should not be interpreted as implying any particular order among or between various steps disclosed herein unless and except when the order of individual steps is explicitly described.

Accordingly, embodiments described herein can be understood more readily by reference to the following detailed description, examples, and figures. Elements, apparatus, and methods described herein, however, are not limited to the specific embodiments presented in the detailed description, examples, and figures. It should be recognized that the exemplary embodiments herein are merely illustrative of the principles of the invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9. All ranges disclosed herein are also to be considered to include the end points of the range, unless expressly stated otherwise. For example, a range of “between 5 and 10” or “5 to 10” or “5-10” should generally be considered to include the end points 5 and 10. Further, when the phrase “up to” is used in connection with an amount or quantity; it is to be understood that the amount is at least a detectable amount or quantity. For example, a material present in an amount “up to” a specified amount can be present from a detectable amount and up to and including the specified amount.

Additionally, in any disclosed embodiment, the terms “substantially,” “approximately,” and “about” may be substituted with “within [a percentage] of” what is specified, where the percentage includes 0.1, 1, 5, and 10 percent.

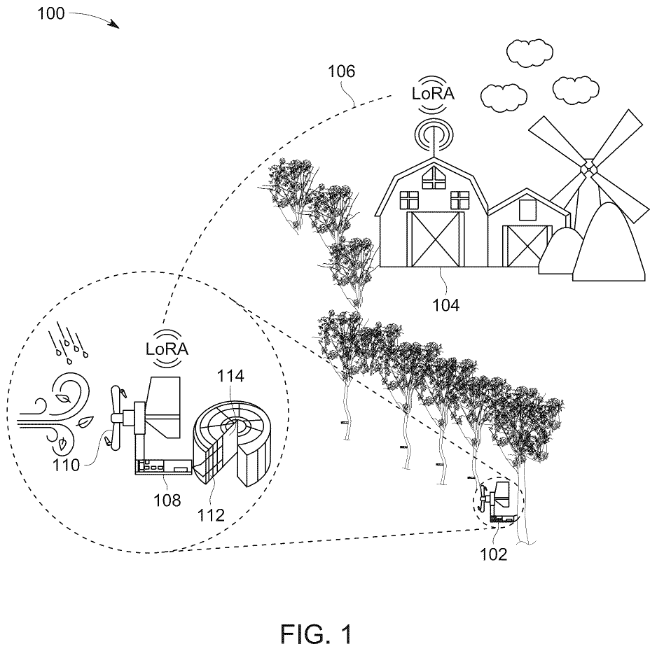

Described herein are biocompatible monitoring and sensing systems which overcome problems and/or deficiencies in prior or conventional designs or implementations. According to some aspects, there is provided a wind-powered, low maintenance, battery-free, biocompatible, implantable, tree wearable, and intelligent sensing system (referred to herein as a biocompatible sensing and/or monitoring system, sensing and/or monitoring system or otherwise “IoTree”) to continuously capture the signals inside the body of a living tree and to infer its water and nutrient levels as illustrated in FIG. 1. A biocompatible sensing system can and/or is configured to monitor nutrient and/or water levels in one or more trees and/or plants by continuously measuring impedance variations inside the tree and/or plant's xylem, which is integral in transporting nutrients and water from the root to the upper parts of the trees and/or plants. The biocompatible sensing system can further collect data or information about the nutrient and/or water levels of a tree and/or plant in real-time, and generate and/or store a dataset corresponding to the nutrient and/or water levels, for instance a time-series data set. In some aspects, the collected and/or generated data can be locally or temporarily stored, for instance at a sensor-transmitter unit as a part of the biocompatible sensing system. The collected and/or stored data can then be compressed before being transmitted to a base station at up to about 1.8 km (approximately 1.1 miles) of distance away. In some other embodiments, the collected data can be transmitted to a base station at up to about 2 km, at about up to 4 km, or up to 6 km away. In some other aspects the collected data may be transmitted over even further ranges.

According to some embodiments, the biocompatible sensing system can be powered by a natural source (e.g., wind power, solar power) and can be controlled by a lightweight and adaptive block-based intermittent computing algorithm. A battery-free sensing system for trees can thus provide for ultra-long multi-day missions, low maintenance, and a lower ecological impact since plugged-in or battery-powered devices cannot afford dense, long-term deployment.

In some embodiments of the present technology, biocompatible and implantable sensors are provided that can be injected or implanted into the trees to measure the water and ion levels using an impedance sweeping technique. In some aspects, a biocompatible sensing system is configured as a battery-free plant-wearable system having biocompatible and implantable sensors for a living tree or plant to continuously monitor nutrient uptake. As will be appreciated, the biocompatible sensing system can monitor any number of information points about a tree or plant in real-time, and further either continuously or in batch with a predetermined run time. According to some aspects the system includes a biocompatible fiber-based sensor that can be implanted inside of the xylem of a living tree or plant. In some further aspects the system can incorporate a plurality of sensors implanted or otherwise disposed in the xylem of a tree or plant. In some aspects the one or more sensors can be a part of and/or in electrical or operable communication with a sensor-transmitting unit or component. In some aspects a sensor is able to monitor up to 5 and/or up to 10 levels (e.g. of concentrations) of NH₃ and/or K₂O nutrients with the accuracy of 91.08% and 90.51%, respectively. As will be appreciated, with real-world implementation, estimated nutrient and water level(s) can be used to indicate normal, lack of water/nutrient, and overwater/nutrient in real-time. The sensed and/or monitored data can then be wirelessly reported and/or transmitted to a base station at a distance away from a sensor-transmitting unit or component, for example up to kilometers away. According to some aspects, the entire system can be powered by natural sources, such as wind energy and/or solar energy for example. In one instance, to cope with the unpredictability of wind energy, a block-based computing method can be implemented to allow and enable the system to fully utilize the energy with minimum memory overheads in operation.

Referring briefly to FIG. 1, a biocompatible sensing and/or monitoring system 100 is illustrated in accordance with embodiments of the present technology. In some aspects, a sensor transmitter unit can be employed at a plant and/or a tree. For instance, the unit may be attached to the tree so that the unit is in operable communication therewith. The sensor-transmitter unit 102 can comprise, among other components, one or more sensors 112 that are implanted into a pant or tree 114, for instance the xylem. Sensor-transmitter unit 102 can further comprise one or more energy harvesting components 110 which may incorporate a plurality of sub-components to capture and/or harvest and store natural energy to be used by the sensor-transmitter unit 102 or the system 100. Further, sensor-transmitter unit 102 can incorporate one or more circuits, data processing units, and/or communications components 108. Such components can process, optionally store, and transmit one or more data sets corresponding to information about the tree and/or plant at a distance 106 to a base station 104. It will be appreciated that base station 104 may be one or more user devices, servers, and/or storage units which can be connected to a network.

As described further herein, the biocompatible sensing system is experimentally evaluated through in-lab and real-world deployment for 30 days. The experimental results show that the biocompatible sensing system is able to provide sufficient and accurate measurements every day with the system running continuously. The system has been shown to report about 558 measurements on average a day with a distance of up to about 1.8 kilometers, without requiring any batteries or maintenance over the time period.

As will be appreciated, conventional apparatus, methods, and systems have various inefficiencies and implementation challenges which are overcome by the present technology. For instance, many sensors have been developed to monitor human physiological signals, but very few target tree health monitoring. Building robust and biocompatible sensors for tree and/or plant monitoring faces multiple challenges from design to implementation as the characteristics of the signals from inside the body of the living tree are not well-known. Further, signals captured from the tree's body, i.e. from within the tree or plant are often weak and buried under other noise. Therefore, building and implementing battery-free, low-power, and reliable sensing hardware and software that are able to provide reliable measurements continuously is a difficult task. Also, just like humans, trees grow over night. Thus, existing solar-based battery-free computing systems are not suitable for tree health monitoring. In the present technology, it has been found that wind energy is a good resource, but developing a wind-based battery-free system is challenging because of the frequent power failures caused by unpredictable wind availability. Further the biocompatible sensing system's tasks, including sensing, computing/compression, and long-range communication/transmitting, can consume different or variable power levels. Accordingly, in some aspects, the computing technique employed by the system needs to be able to adaptively update its run-time schedules to fully utilize the harvested energy and complete user-based or end requirements.

Accordingly, aspects of the technology described herein provide at least the following advantages: a biocompatible fiber-based sensor to sense and monitor water and/or nutrient levels inside a tree/plant body; a battery-free and low-power sensing algorithm is derived and configured to measure signals inside the tree body reliably under multiple or changing environmental conditions; a block-based intermittent computing algorithm is built and implemented allowing and enabling the biocompatible sensing system to fully utilize harvested energy and perform tasks with the minimum required memory and energy; the biocompatible sensing system opportunistically performs sense, data compression, and long-range communication without requiring a battery and/or maintenance over an operational time frame; and the biocompatible sensing system is validated with in-lab and in-the-wild environments confirming that the biocompatible sensing system can obtains at least 91.08% and 90.51% of accuracy in measuring up to 10 levels of nutrients, NH₃ and K₂O, respectively. When tested with Burkwood Viburnum and White Bird trees in the indoor environment, the biocompatible sensing system data strongly correlate with multiple soil stimuli (e.g. watering and fertilizing) events. When deployed in the wild for 30 days the biocompatible sensing system results show that the system is able to provide sufficient measurements every day and/or continuously over the time-period. In some aspects, the system can report 558 measurements a day with a distance of up to 1.8 kilometers without requiring any batteries or maintenance.

Additionally, the biocompatible sensing system platform and/or system and/or method has potential to be used in precision agriculture, global warming, crop monitoring, plant physiology research, plant disease monitoring and pest control, and others. The hardware design, firmware, and software libraries of biocompatible sensing system can be used across multiple applications.

In some aspects, a biocompatible sensing system relies on the unique chemo-electrical relationship between water, nitrogen (N), and potassium (K) ion levels inside the xylem sap of trees and the measured impedance levels measured by the sensors of the system.

According to some embodiments, a system for monitoring nutrient levels in the body of a tree are provided, the system comprising: a sensing component comprising a sensor implanted into the tissue of the tree to generate a dataset that corresponds to information about the tree, a communication component to transmit data, the data comprising at least the generated dataset, and a base station to receive the data. The system can further comprise an energy harvesting component to obtain energy from one or more natural resources and transfer power to at least a portion of the system, and further the system can comprise a natural resource indicator component to optimize the obtained energy. In some instances, the sensor comprises one or more biocompatible fiber electrodes and accordingly, the sensing component can generate one or more frequency signals associated with, for example, a tree. Additionally, the sensing component can generate one or more of an impedance or impedance profile for a tree based on the generated dataset. In some instances, a water and/or nutrient level corresponding to the tree is determined from the impedance or impedance profile. In some instances, the sensor is implanted in the xylem of the tree. In some instances, the impact of environmental noise is reduced or minimized. In some instances, at least a portion of the components operate in response to a block-based intermittent computing model which is configured to control any aspect of the operation of the biocompatible sensing system.

According to some even further embodiments, a computer-implemented method for monitoring nutrient levels in the body of a tree is provided, the method comprising: implanting one or more sensors into a tree body, capturing one or more signals from the tree to generate a dataset corresponding to the tree, determining an impedance and/or impedance profile for the tree, and transmitting the at least one of the dataset, the impedance and/or impedance profile to a base station. In some instances, the method comprises compressing the at least one of the dataset, the impedance and/or impedance profile prior to the transmitting. In some further embodiments, the method comprises generating one or more frequency signals. In some instances, any of the signals may be processed. Further, in some embodiments, a method can further comprise harvesting energy from one or more natural resources. In some other instances, any steps of the method may be carried out continuously, for example transmitting may be carried out continuously.

According to some even further embodiments, a system is provided having a computer storage medium storing computer-useable instructions that, when used by one or more computing devices, cause the one or more computing devices to perform operations comprising: capturing one or more signals from the tree to generate a dataset corresponding to the tree, determining an impedance and/or impedance profile for the tree, and transmitting the at least one of the dataset, the impedance and/or impedance profile to a base station. In some instances, operations can further comprise at least one of harvesting energy from one or more natural resources, generating one or more frequency signals, and/or processing captured signals.

Aspects of the present technology rely on ion ratios inside the transport systems of living plants/trees. In some aspects systems and methods described herein focus on water, nitrogen, and potassium monitoring since they are confirmed as the most important substance in tree development. Note that other mineral elements such as phosphorus (P), calcium (Ca), magnesium (Mg), sulfur (S), and sodium (Na) also have vital factors in facilitating the metabolism. However, we reserve the studies of these minerals for future work. Specifically, tree roots take up nutrients from the soil by absorbing their ions. Transceptor proteins of the roots regulate the nutrient uptake through an absorption process, which is caused by the difference between the inside and outside ion concentrations of the roots. After being taken by the root through the nitrogen fixation process, nitrogen is stored and transported inside the tree under the form of nitrate (NO₃⁻), nitrite (NO₂⁻), or ammonium (NH₄⁺). The optimal nitrogen for healthy trees is maintained from 3% to 4% in their tissues. Similarly, the root works together to maintain the optimal concentration of potassium (K⁺) at 80-100 mM inside the tree cells. These nutrients are taken from the soil and transported inside the xylem to maintain the balance of tree cells.

As the roots uptake nutrients from the soil and transport their ions through the xylem, the impedance changes accordingly to each ion's concentration. Previous vitro experiments showed a strong correlation between the measured impedance spectra (from 1 Hz to 1 MHz) and a wide range of NO₃⁻ ion concentrations. They also confirmed that the resistance-capacitance parallel circuit model can be used to explain the behavior of ionic charges in ionic conductors. Further studies also found similar results on the relationship between the concentration of nutrient ions (K⁺, NH₄⁺) and impedance spectra of its medium. This relationship will be later confirmed in examples and experiments described herein.

By measuring the impedance level inside the living tree, we could infer the ion level, which can be used to indicate the tree's health. However, multiple hinders need to be overcome to build a reliable and practical sensing system. First, a biocompatible solution needs to be developed as monitoring impedance inside the tree requires implanting the sensor into the tree body. While biocompatible sensors have been developed for human, their applicability in tree sensing is unclear. Second, plugged-in or powered devices are not suitable for large-scale and long-term deployment in farm settings. The battery-free system is the most suitable solution, but existing battery-free solutions focus on solar-based setup; therefore, their concepts are not suitable for overnight and continuous sensing. In the following sections, we will describe the proposed system to address the above challenges, followed by the detailed design, implementation, and evaluation of each subsystem with in-lab, indoor, and outdoor experiments.

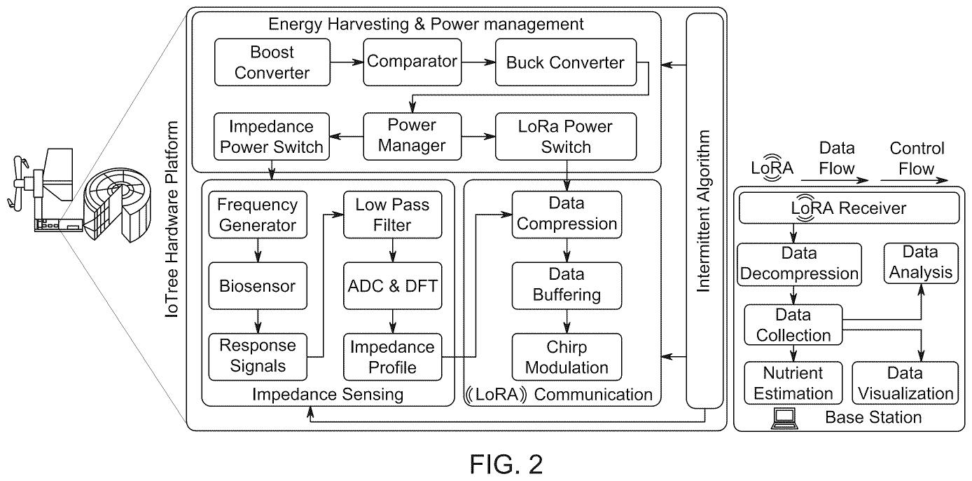

At a high level the biocompatible sensing and monitoring system implemented and configured as follows. First, the system can capture the signals inside the tree's body; hence, the sensors can be implanted into a tree or plants' tissue. Second, the system can be configured to require the least amount of maintenance since it may be deployed on a large scale, and frequent charging or replacing batteries is not practical in an agriculture setting. Thus, the system in some instances can be completely battery-free and can utilize harvested energy from natural sources. Lastly the system can report or transmit data at a long distance. At a high level, the biocompatible sensing system is illustrated in FIG. 2, and the various system components are further described herein.

Biocompatible Sensor and complementary Sensing Technique. The biocompatible sensing system utilizes impedance-based biocompatible sensors that can include two or more fiber electrodes made by biocompatible materials to ensure the sensors are able to live within a plant or tree. The sensing circuit generates sweeping frequency signals and measures the responses to calculate the impedance. Since the signals are weak, an amplification circuit has been designed to cope with the impact of environmental noises. We use multiple pairs of sweeping frequencies and the value calculated from Discrete Fourier Transform (DFT) to form impedance profiles that are used to infer the water and nutrient levels.

Energy Harvesting and Power Management. In some aspects, the biocompatible sensing system can operate fully based on energy harvested from the wind. A lightweight wind energy harvester is implemented, including a wind indicator that can maximize harvested power for operation. Additionally, wind behaviors can be analyzed in a given area where a system is deployed and optimize the system components and/or operation schedule, thereby ensuring that the harvested energy is sufficient to perform the systems tasks over a given period of time.

Block-based Intermittent Computing. According to some aspects, a block-based intermittent computing approach is implemented that is more suitable to wind-power compared to other state-of-the-art intermittent computing solutions (i.e., task-based and checkpoint based approaches). Accordingly, the block-based approach utilized by the system adaptively changes the number of blocks and the size of each block to minimize the memory overhead and wasted energy, which can in some instances be based on variable environmental conditions or factors. Additionally, in some aspects, after a reboot, for example after a failure or some other event, the biocompatible sensing system can recover from the last executed block in a given task instead of restarting from the beginning of that task.

Long-range Communication. Since the biocompatible sensing system can be deployed on farm settings, it can be enabled and/or configured to transmit data to one or more base stations accurately and reliably. The average farm size in the U.S. is 444 acres; hence, the typical communicating distance requirement for a device is 0.59 miles (0.95 km). In some aspects a LoRa communication is implemented in the system to enable reliable, low-noise, and low-power communication. Accordingly, the biocompatible sensing system can report and/or transmit data continuously to a base station, for example at 1.8 kilometers away, or up to 2 kilometers away.

Sensor Design. As a biocompatible sensing system's sensors are implanted inside the tree, they need to be small and biocompatible in operation within a plant or tree, for example. Accordingly, in some aspects, a fiber-based impedance sensor is utilized. With respect to an impedance sensor, fiber-based impedance sensors are microscale, soft, and lightweight conductive fibers, which are made from reduced graphene oxide/polyurethane/silver nanowires (rGO/PU/AgNWs). Soft microfibers can be fabricated by using a wet-spinning method. First, the rGO/PU/Ag-NWs gel microfibers are formed via a wet-spinning method. Subsequently, the gel is reduced by the ascorbic acid solution to generate conductive soft rGO/PU/AgNWs microfibers. Lastly, this substance is taken out and dried at 120 °C for 2 hours in a vacuum oven. The sensor, therefore, provides high biocompatibility and good physical stability. In addition, as itis soft and flexible, implanting these electrodes into the tree body can be done with ease.

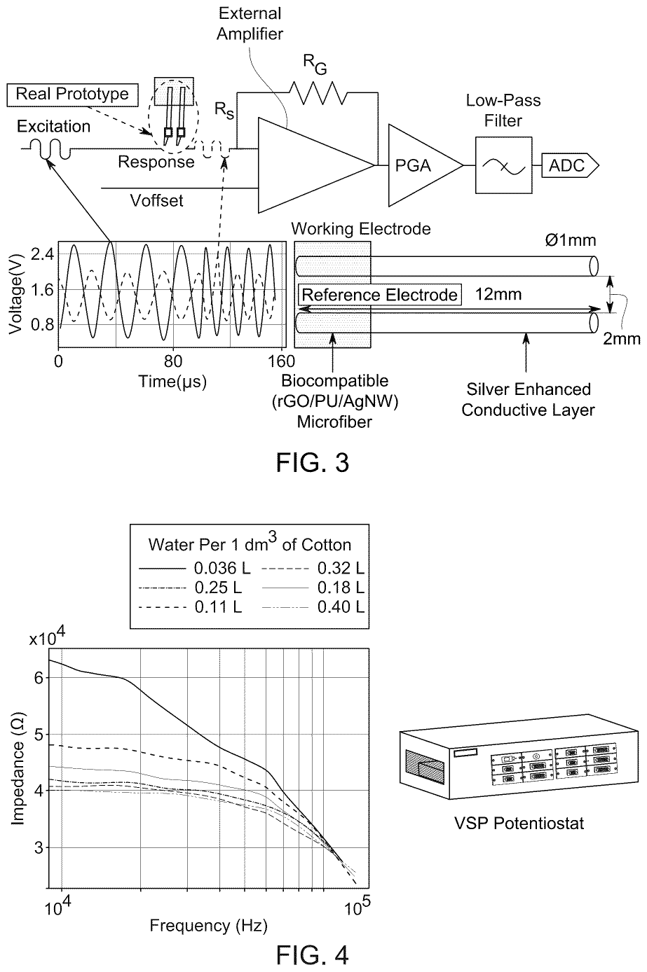

In some instances, the sensor can incorporate one or more electrodes, for instance two conductive soft rGO/PU/AgNW microfiber electrodes, as depicted in FIG. 3. The microfibers are directly used as a working electrode (WE). To develop the reference electrode (RE), the rGO/PU/AgNW microfibers are coated in Ag/AgCl. Then, the WE and RE microfiber electrodes are aligned in parallel with a distance, for example a distance of 2 mm between each other to form a soft impedance sensor. The rGO and AgNWs can be embedded in PU. As will be appreciated, the PU presents unique chemistry with a molecular structure similar to that of human proteins. Thus, the impedance sensors made from rGO/PU/AgNWs microfibers can provide high biocompatibility for living inside of trees or plants.

In-lab Sensor Sensitivity Validation. Sensors are validated using a VSP Potentiostat device. FIG. 4 shows various impedance profiles measured by the device. The reading from sensors shows that they can distinguish the different amounts of water applied to the same volume, which means the sensor is sensitive and ready to work. However, a VSP Potentiostat is heavy (9 kg), big (95 mm×435 mm×335 mm), and expensive and not suitable for outdoor tree monitoring.

Sensing Principle. According to some aspects, an inexpensive, low-power, and small-sized sensing circuit to replace a VSP Potentiostat with the working principle illustrated in FIG. 3. In particular, to measure impedance, an excitation voltage signal U is applied on one electrode, and the resulting current I is measured at the other electrode. Assuming an alternating current with frequency f applied to the first electrode (the applied voltage is v(t)=U₀ cos(ωt)) creates current i(t)=I₀ cos(ωt+φ) in the second electrode, where (φ) is the phase shift between v(t) and i(t); ω=2πf. Impedance Z can be presented in polar form: Z=|Z|e^{jφ}=|Z|(cos(φ)+j sin(φ)), where |Z| represents the ratio between voltage amplitude and current amplitude. Another form representing impedance uses the Cartesian form: Z=R+jX, where R is real value, and X is imaginary value. The transformation from polar form to Cartesian form is calculated by R=|Z|cos(φ), and X=|Z|sin(φ), while the transformation from the Cartesian form to the polar form is calculated by |Z|=√R²+I², and φ=tan⁻¹(I/R).

Signal Processing. Due to the high impedance of the monitoring object (from 200 KΩ to 1 MΩ), the response signals are feeble. Therefore, these signals are amplified by the chain of two amplifiers before being filtered and sampled. This chain includes one external amplifier and one on-chip PGA. Both amplifier gains can be configured by adjusting the value of RG and GA. Consequently, the signals at the input pin of the Low-Pass filter can be calculated by Vr=(RG/RS)·GA·Vx before being sampled by ADC, where RG is gain resistor value, RS is object resistance value, GA is PGA gain factor, and Vx is excitation signals. For each frequency, the impedance Z(f) is calculated by DFT using equation: Z(f)=P1023n=0 (x(n)(cos(n)−j·sin(n))), where Z(f) is the impedance at frequency f, x(n) is the sampling result at the time n. As a result, we can calculate the real and imaginary values from Z(f). Multiple-frequency sweeping technique is used to ensure the diverse characteristics of the substances retrieved from the response signals.

Sensing Circuit. We build the circuit based on impedance analyzer chip AD5933. We select this Integrated Circuit (IC) because of its reliability and low power consumption (11 mA in operation mode). The IC includes a frequency generator that modulates 1 Hz to 1 MHz excitation signals. The response signals will be sampled by 12-bit ADC and calculated by Discrete Fourier Transform (DFT) supported by hardware. We also design an external amplifier that works along with an internal programmable gain amplifier. After sweeping one specific frequency, we can get four bytes (two bytes for real value and two for imaginary value) resulting from DFT processing. Every four bytes are stored in the FRAM and managed by the block-based computing algorithm.

TABLE 1
Compression ratio for IoTree's data

| No. | No. | LZW | Huffman |
|---|---|---|---|
| Measurements | Bytes | Ratio | Ratio |
| 1 | 64 | 0.72 | 1.55 |
| 2 | 128 | 0.61 | 1.27 |
| 3 | 192 | 0.56 | 1.09 |
| 4 | 256 | 0.55 | 1.02 |

Data Compression. Due to the high energy consumption of the communication task, data compression is needed for the biocompatible sensing system's communication. ZLW can be adopted for data compression reliability. ZLW avoids the overhead of sending a dictionary used to decode data like Huffman coding. More importantly, ZLW can work well if the input data is sufficient. Our implementation of impedance profile measurement includes 64 bytes, therein 16 sweeping frequencies, each occupying four bytes. Our observation on data collection is that one measure may be slightly different from others if the data are collected close in time. If we send raw bytes data, the compression algorithm cannot work well because each byte entry value distributes in a range of from 0 to 255. However, most differential data between the two measures are distributed from 0 to 50, so the room for data compression is intensified. The compression ratio is defined as output size divided by input size, and Table 1 shows that ZLW outperforms Huffman with the biocompatible sensing system's sensing data. The more data that can be compressed, the better performance is obtained. Accordingly, a number of measurements of the system can be determined based on application need and MCU's memory size.

Long-Range Wireless Communication. The biocompatible sensing system supports long-range communication capability to ensure that the system meets the requirement of agriculture deployment. Various solutions for communications may be implemented, including NBIoT and LTE-M, Wi-Fi, Backscatter, LoRa backscatter, and LoWAN. NB-IoT and LTE-M provide low-power and long-range communication allowing the system to upload data directly to the internet. However, current 5G NB-IoT and LTE-M prototypes are only available from a few vendors, and they consume high power and require to be connected to a power outlet. In addition, they do not support custom-built implementation. More importantly, they are not free of charge. Backscatter and LoRa backscatter are promising concepts. They are also considered as the most suitable designs for the biocompatible sensing system. However, Backscatter supports a short distance of communication while LoRa-backscatter faces challenges in concurrent communications. Wi-Fi and Bluetooth 4.0 are two other popular techniques that can be used for the biocompatible sensing system. However, Wi-Fi consumes high power while Bluetooth only provides a short distance of communication. Modified Bluetooth 4.0 requires heavy engineering effort.

In some aspects, LoRa Wan can be implemented as the main communication protocol for the biocompatible sensing system to ensure the system's usability, reliability, and practicality. While LoRa consumes more power than state-of-the-art low-power long-range communication such as LoRa Backscatter or modified Bluetooth 4.0, our hardware design can be easier integrate with LoRa Backscatter or Bluetooth 4.0 hardware if they are available in the market. A Lora module can be implemented using RFIC SX1276. The IC operates at frequency 915 MHz and consumes 100 mW at a maximum transmitting power of 20 dBm. In an ideal environment with the light of sight (LOS) conditions, in some instances, LoRa communication can support communication distance up to 3 km for the system.

Battery Free Design. Multiple energy harvesting methods can be implemented in the present system including solar-based, piezoflag-based, heat-based, and wind-based. The solar-based method provides reliable and constant power, but its performance may be reduced when the light is blocked by the tree. In addition, it does not work during the night. Heat-based energy-harvesting prototypes with CM² size does not provide sufficient energy to power the circuit. The flag-based approach does not provide efficient power (average of 3.1 mW) within the same period of time compared to the turbine-based approach (average of 8.0 mW). Furthermore, the piezoelectric flags ($160) are much more expensive than the motor for the wind turbine ($2). Therefore, wind energy is the most suitable and practical solution.

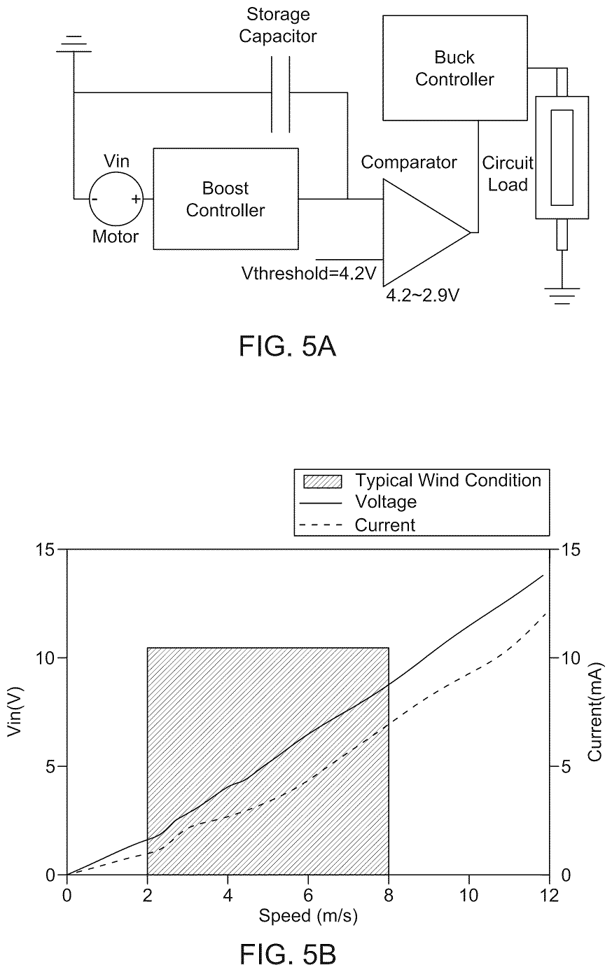

An energy harvesting circuit as shown in FIG. 5(a) can be implemented in the system. The harvesting circuit includes a boost controller and a buck controller, optimizing the conversion performance from observed wind power to usable energy. The boost controller harvests energy from the lowest power (2.2 V, 1.1 mA at 2.2 m/s) up to the highest power (12 V, 13.7 mA at 12 m/s) of the motor to charge a storage capacitor while typical wind speed in the testing area is from 2 m/s to 8 m/s as shown in FIG. 5(b). This capacitor plays a role as a static energy buffer to power the circuit. When the storage capacitor's voltage reaches the defined threshold, the comparator triggers the buck controller to power the primary computing circuit.

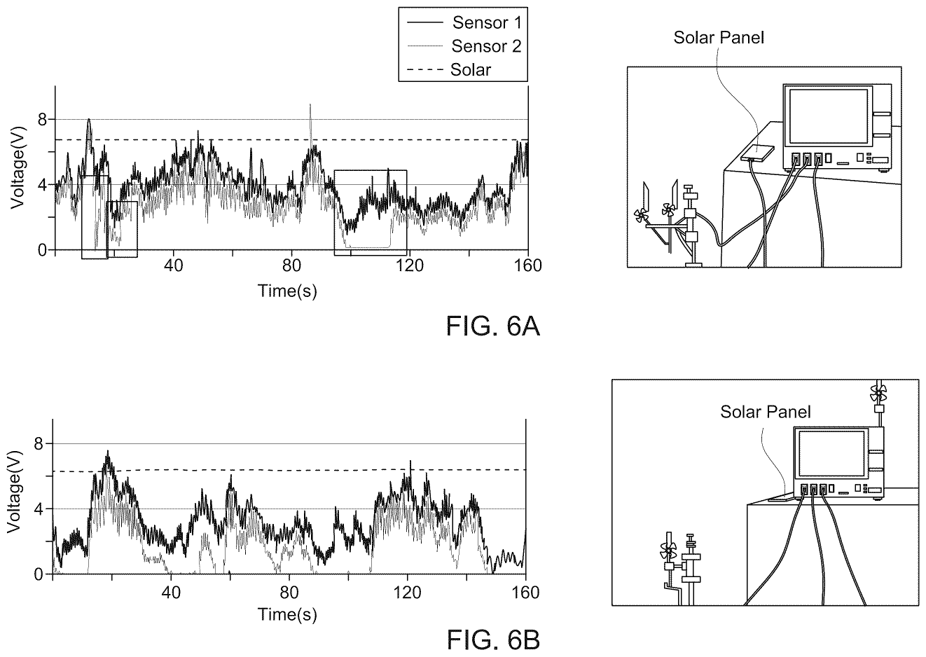

Wind energy is unpredictable and non-uniformly distributed in any geographical area. To validate this theory, two harvesting circuits are operated at a distance of 5 cm and 1.5 m and measure voltage from these two circuits. The solar energy at these locations is also measured. FIG. 6 (a) and FIG. 6 (b) show the capacitor voltages from two wind harvesters that are 5 cm and 1.5 m apart, respectively. As can be seen from these figures, the harvested wind energy is different in both cases, while the energy obtained from solar power is almost constant.

Block-Based Computing. A common objective of designing a battery free system is to maintain forward progress and data consistency. Although efforts have been made to deal with intermittent characteristics of harvested energy in batteryfree systems, existing works focus mostly on solar-based systems. Unfortunately, as wind's unpredictable and non-uniformly distributed behavior induces non-continuous harvested energy, these proposed solutions (i.e., checkpoint approaches, and task-based approaches) are not well-suited to wind-powered batteryless systems due to the following reasons. First, the checkpoint based approach takes snapshots of the entire system, including stack functions, local variables, global variables, and others before power failure, and then restores these snapshots after the power returns. While this technique is extremely efficient in energy utilization since the code will be resumed at the precise location, they result in much memory overhead due to storing, restoring, and rebooting efforts. Second, the task-based approach stores the states of executed tasks before power failure; the unfinished task has to be re-executed from the beginning of the task. By doing so, the system only needs to store/restore the states of the completed tasks instead of the entire system. This method is efficient in minimizing the memory overhead. It is, however, inefficient in energy utilization because if the power fails in the middle of the task, that task is going to be re-executed from the beginning.

Considering a capacitor size of 6 mF, the maximum harvested energy is 27 mJ. The system excuses three tasks T1, T2, and T3 which consume 10 mJ, 0.5 mJ, and 36 mJ, respectively. In task-based approach, the system will finish first two tasks and T3 will never finish because the maximum energy available (27 mJ) is less than T3 requirement (36 mJ). Applying the checkpoint approach requires significant additional energy since the system has to take a snapshot of the entire system state every time the power goes down. In some aspects of the biocompatible sensing system, checkpointing can consume from about 0.6 to 2.6 mJ of overhead.

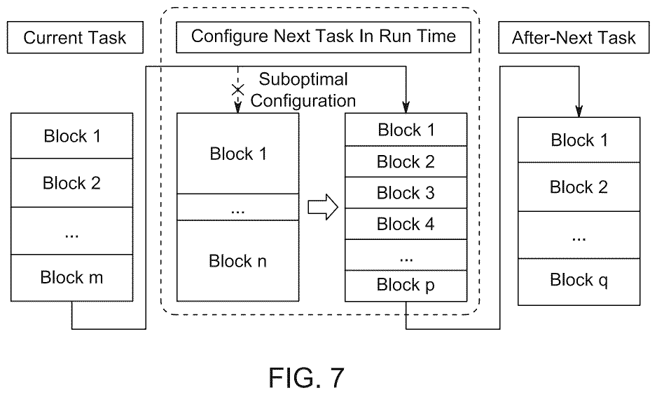

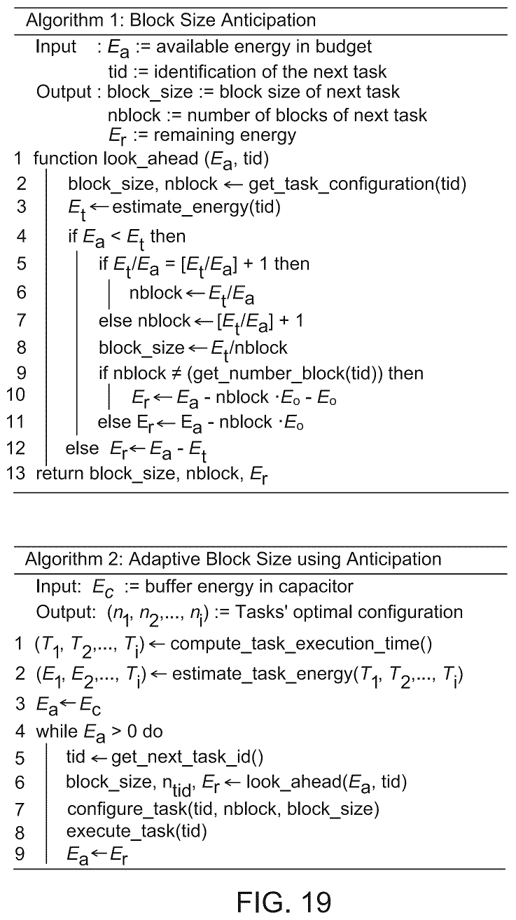

In some instances, a blocked-based approach is implemented to overcome the aforementioned limitations. The key idea is to boost energy utilization by adaptively changing the amount of codes to be executed before power failure, and to minimize overhead memory by check-pointing only replicas of the system's snapshots (i.e., pointers). In particular, the task is divided into multiple blocks whose sizes can be adaptively updated during run-time depending on the energy availability. FIG. 7 illustrates an example of how block-based approach works during run-time. The biocompatible sensing system computing system strives to obtain the most optimal number of blocks for the tasks in terms of wasted energy. Specifically, a sequential optimization pipeline can be implemented, including a theoretical optimization in compile-time and an empirical optimization in run-time. For the theoretical optimization stage (i.e., compiling time phase), the hypothesized solution is the trivial solution based on the power consumption ratio among three tasks computing from the machine code distribution of each task on FRAM. Afterward, the hypothesized solution is served as initial block numbers for the biocompatible sensing system in the empirical optimization stage, which only occurs when the system is deployed (i.e., during its run-time phase). The empirical optimization stage uses Alg. 1 of FIG. 19 as a configuration method to adjust the hypothesized parameters to be a more precise solution (i.e., near-optimal block size and the number of blocks) in the system's run-time (Alg. 2 of FIG. 19). This process returns the most optimal set-of-integer of block size and the number of blocks.

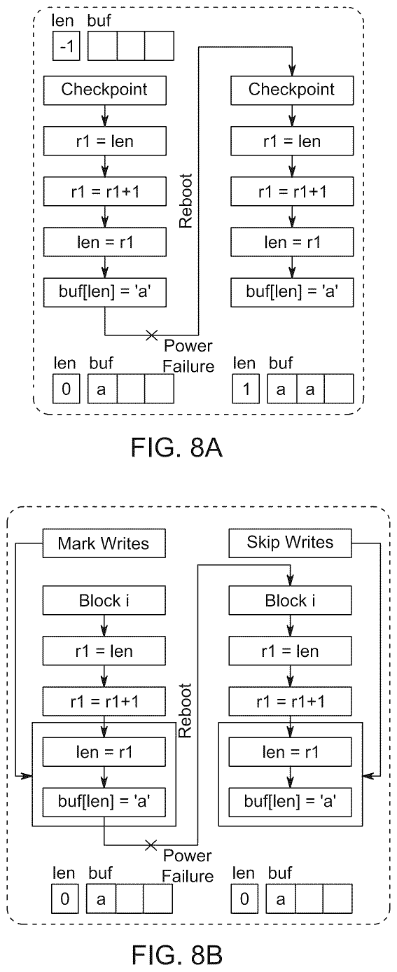

Forward Progress and Data Consistency. Block-based approach maintains forward progress by saving the task pointer whenever the system jumps into the task and saves the current block pointer of that task. To maintain data consistency, a buffer manager can be implemented, which prevents multiple writes into output buffers when a task resumes from the same block multiple times. FIG. 8 (a) shows a typical idempotent violation, which produces two different results when executing the same code. FIG. 8 (b) illustrates how buffer manager effectively controls the “write” and “skip write” data, avoiding data inconsistency.

Block-based Library Implementation. The block-based run-time library is implemented using the C programming language, the ideal language in embedded system development. The library aims to give users a set of Application Programming Interface (API) to develop an intermittent application effortlessly. These APIs include setting up tasks and blocks. The user initially needs to create tasks by using create_task(name,&task_id), connect them by set_transition(task_id1, task_id2), and set input-output buffers by set_buffer(task_id, buffer,buffer_type) for each task. Before setting buffer for a task, memory needs to be allocated for that buffer by alloc_buffer(size). Non-volatile memory is the crucial component to save a program state before a power failure so that the application can continue from that failure point. Creating buffers with help from the non-volatile memory manager provided by the run-time library enables programmers to write an intermittent program efficiently. The library allows them to focus on their algorithms and let the run-time library manage all global non-volatile variables, which are potential causes of data inconsistency. Next, the user must create block handlers to manage block control and set these block handlers as well as the number of blocks for each task by using set block handler (task_id, block_func, number_blocks). The system will execute the setup code only once at the first power cycle, then run as scheduled unless programmers want some changes at the run-time, such as re-wiring task transitions for special cases.

TABLE 2
Power consumption and typical execution time for IoTree's tasks

| | Idle | Sensing | Computing | Transmitting |
|---|---|---|---|---|
| Power (mW) | 1 | 62 | 6 | 245 |
| Duration (ms) | 0.3 | 170 | 80 | 150 |

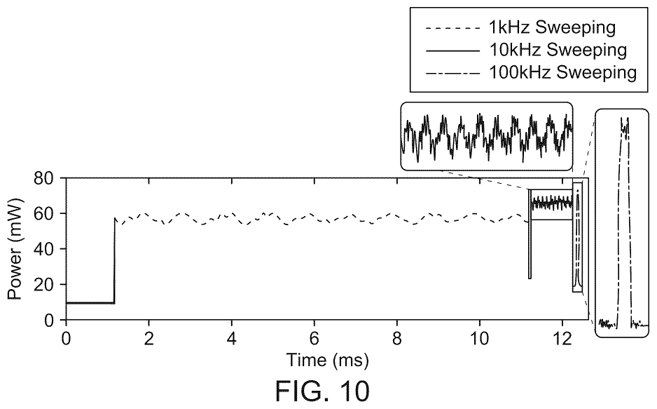

Block-based Implementation. The biocompatible sensing system includes three main tasks: sensing, data compression, and communication; each task is adaptively divided into blocks using the block-based run-time library. The power consumption and typical execution time of each task are shown in Table 2. A task must be accomplished before the system proceeds to the next task, and each of them reads the input from the previous task and produces the output for the subsequent one. Due to the imperfection of electronic components, even the operational power of a task fluctuates over time, leading to many difficulties in obtaining optimal block size and number of block. Considering the sensing task as an example, our system captures the capacitance response created by sweeping 16 frequencies signals. FIG. 10 illustrates the energy consumed at frequency 1 kHz, 10 kHz, and 100 kHz. The energy consumes by each frequency is different, and frequent power failures make it extremely difficult for the system to identify the best block size and number of blocks. Fortunately, the variation of the consumed power of each task is predictable. If the system obtains sufficient knowledge of how each task consumes power during its run-time, it will be able to identify the best block size and block number. The starting point of the energy ratio between tasks and the storage capacitor is provided by the user/programmer during compile time. Block size and number of blocks are then obtained automatically during the installation phase of the system.

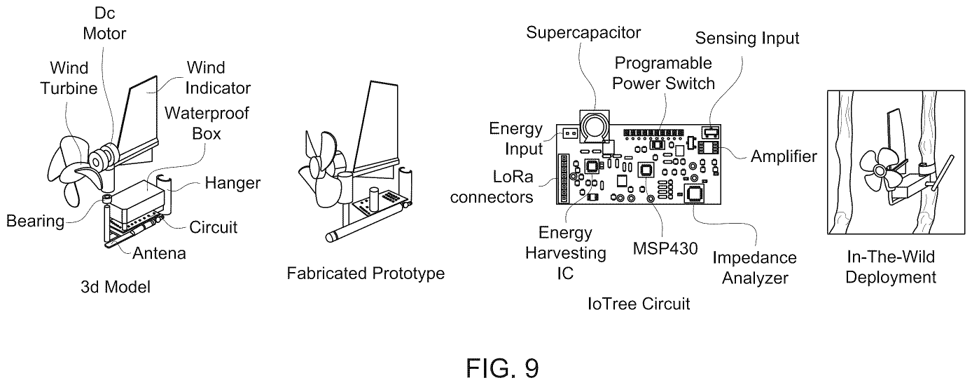

System Implementation. The design, fabrication, and implementation of the biocompatible sensing system can be configured with COTS materials and electronic components as illustrated in FIG. 9. In the following subsections, the 3Dmodel, PCB, implant process, and sensor calibration is described. The process of design and fabricate the impedance sensor is described herein. A software running on a computing device and equipped with a LoRa receiver can be implemented, to continuously receive the signals from system nodes.

3D Model Design and Fabrication. The biocompatible sensing system 3D prototype is designed using SOLIDWORKS. For the system, a φ24 DC motor and wind turbine (φ80) can be implemented for the energy harvester. A wind indicator can also be incorporated that supports 180-degree freedom of rotation to steer the turbine towards the direction of the wind. A hanger is designed at one end of the prototype to help attaching the device to the body of the tree. The vertical pole holding the turbine is a tube that allows the electrical wires to connect the motor and the circuit. This structure will help to avoid blockage caused by twisted wires around the prototype when the wind indicator rotates in one direction for certain cycles. A small bearing is used to reduce friction when the wind indicator rotates around the vertical axis. The whole system, including the circuit, weights 115.85 grams with the size of 145 mm×170 mm.

PCB Design and Implementation. The biocompatible sensing system's circuit as shown in FIG. 9. The circuit has a size of 31.5 mm×65 mm, weights 4 grams, includes two layers with 0.8 mm thickness. MSP430FR2433 (126 μA/MHz, 16KBFRAM, 4 KB SRAM) is the central control unit of the biocompatible sensing system's circuit. To harvest energy at the low wind speed (2 m/s, 1.5V), BQ25570 can be used, which supports a power boost charger to charge a storage capacitor up to 4.2 V from the lower input voltage. The energy harvester also allows the maximum power input of 510 mW that is able to sustain for maximum wind speed (12 m/s, 13.7 voltage, 12 mA, 164.3 mW) as the experiment depicted in FIG. 5. MCU communicates with impedance analyzer circuit via I2C and with RF components via UART. Programmable power switch integrated circuits can be incorporated (TPS22919DCKT IC) to turn on or turn off different circuit components according to the requirement of the current task to force the component to go to sleep if they are not performing any task. The amount of energy buffer is calculated by Ec=(½)·C·(V2max−V2min), where Vmax is the upper bound voltage to trigger starting of the circuit and Vmin is the lower bound voltage where the circuit turns off.

Implanting Sensor. First two small holes are drilled on the tree with a depth of three millimeters, then a portion of mixture 500 mg catalase and 50 ml conductive gel is put into these holes to avoid the reduction in conductivity level. The catalase prevents the defense mechanism of the tree, which will lead to compartmentalization and reduced sensor accuracy while conductive gel increases conductivity and moisture between two electrodes and tree xylem. Two sensor electrodes are placed into two holes and shield these holes with adhesive tape to make sure that no air can come into the holes.

Sensor Calibration. A compact model can be implemented to map the measured impedance profile to the corresponding water and nutrient levels. For each measurement, an impedance profile vector is obtained of k+1 elements, \vec{x}_{raw}=(1, x₁, x₂, . . . , xₖ); each element from x₁ to xₖ respectively represents for the impedance being measured at each frequency, ranging from f1 to fk (k=16 in one example implementation). Depending on each input impedance profile \vec{x}_{raw}, the corresponding predicted nutrient level, ŷ, will be calculated as follows Eq. 1:

$$\hat{y} = {\sum }_{i = 0}^{k} {β}_{i} {x}_{i} + {\sum }_{i \neq j \neq 0}^{k} {β}_{i , j} {x}_{i} {x}_{j} + {\sum }_{i = 1}^{k} {β}_{i , i} {x}_{i}^{2}$$

Leveraging Eq. 1, our data set could be designed into the matrix form as: \vec{y}=X\vec{β}+\vec{ε}, where X is a matrix composing of n vectors in the form of \vec{x}

$$( with \vec{x} = ( 1 , {x}_{1} , … , {x}_{k} , {x}_{1} {x}_{2} , {x}_{1} {x}_{3} , … , {x}_{k - 1} {x}_{k} , {x}_{1}^{2} , {x}_{2}^{2} , … , {x}_{k}^{2} ) ) ,$$

\vec{y} is the vector of nutrient levels corresponding to the impedance profiles, \vec{β}=(β₀, β₁, . . . , βₖ, β_{1,2}, β_{1,3}, . . . , β_{k-1,k}, β_{1,1}, β_{2,2}, . . . , β_{k,k}) and \vec{ε}=(ε₁, ε₂, . . . , εₙ)^{T} is the residual value vector for n measurements. To seek out the most suitable \vec{β}, Iteratively Reweighted Least Squares (IRLS) algorithm can be utilized that allows us to update \vec{β} iteratively. By establishing an objective function in Lp spaces, Lebesgue spaces, for the quadratic regression as follows Eq. 2:

$$arg {min}_{\vec{β}} {‖ \vec{y} - X \vec{β} ‖}_{p} = arg {min}_{\vec{β}} {\sum }_{i = 1}^{n} {\vert {y}_{i} - {X}_{i} β \vert}^{p}$$

where argmin_{\vec{β}} is the minimizer returning coefficient vector, \vec{β}, at which the polynomial is minimum, ∥x∥ₚ is defined by

$${( {\sum }_{i = 1}^{n} {\vert {x}_{n} \vert}^{p} )}^{\frac{1}{p}} ( p ∈ ℝ \vert p \geq 1 )$$

and Xᵢ is the i^{th} row of X. To solve Eq. 2, initial values for \vec{β} and W are obtained. The initial \vec{β}, \vec{β}⁽⁰⁾, is the resulting β from Weighted Least Squares algorithm running on our dataset. The initial diagonal weight matrix W, W⁽⁰⁾, is assumed as the identity matrix Iₙ. In the loop, \vec{β} and W will be automatically updated by:

$${\vec{β}}^{( k + 1 )} = arg {min}_{\vec{β}} {\sum }_{i = 1}^{n} {w}_{i}^{( k )} {\vert {y}_{i} - {X}_{i} β \vert}^{2} = {( {X}^{T} {W}^{( k )} X )}^{- 1} ( {X}^{T} {W}^{( k )} \vec{y} ) and$$

$${W}_{i , i}^{( k )} = {\vert {y}_{i} - {X}_{i} {β}^{( k )} \vert}^{p - 2} .$$

Also, the weighted least-squares error is re-calculated as:

$$e = {\sum }_{i = 1}^{n} {{w}_{i}^{( k )} ( {y}_{i} - {\hat{y}}_{i} )}^{2} .$$

The loop stops when the error converges. Hence, the most suitable \vec{β} can be obtained with the minimum \vec{ε} when the loop ends. Note that the developed model is designed for a single substance (nitrogen or potassium); a more sophisticated learning algorithm is needed to estimate the nutrient levels when multiple nutrients are fertilized simultaneously.

### Experimental Performance Evaluation

In-lab and in-the-wild experiments are conducted to validate the sensitivity and feasibility of the biocompatible sensing system. The experiments are designed to answer the following questions: (1) Is it possible to monitor different water and nutrient levels using the developed sensors?; (2) What is the accuracy of the sensing technique?; (3) Is it possible to monitor how the tree react to the input water and fertilizer in real-time?; (4) Can the system operate completely battery-free?; (5) Is the implemented system reliable for long-term deployment.

### Indoor Experiments.

In Situ Experiments. First the accuracy of monitoring (NH₃ and K₂O) nutrient levels is validated through in-lab experiments by producing different levels of concentrations for each substance ranging from 10 g/L to 100 g/L (i.e., amount of substance over the mixed water). At each level, the measurements are repeated 100 times; 70 measurements are used to build the model (finding 8 as in Eq. 2), and 30 measurements for testing our model as in Eq. 1.

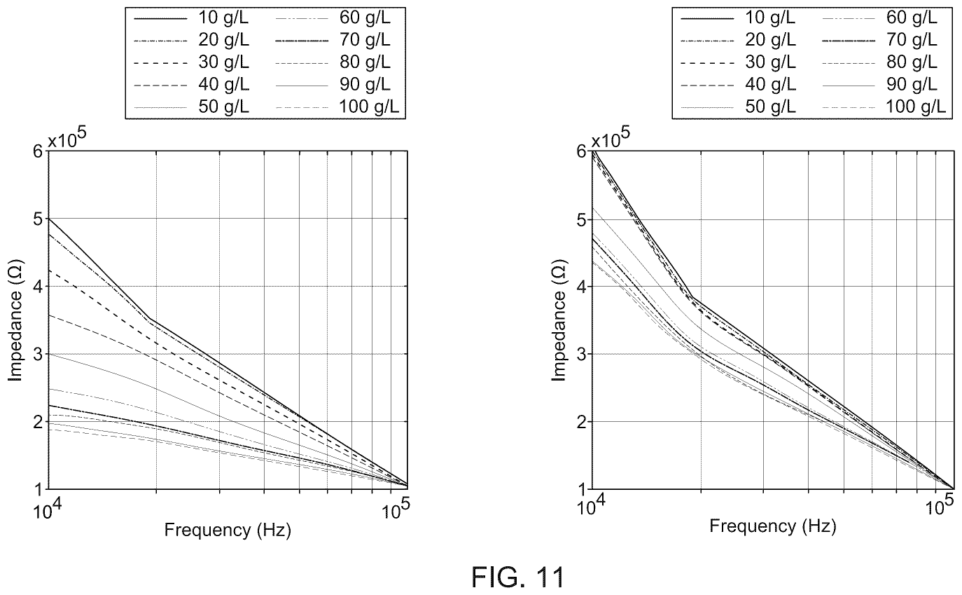

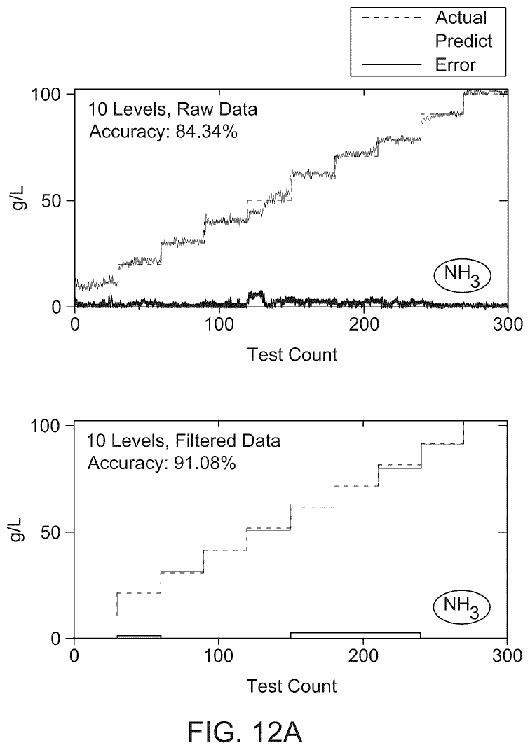

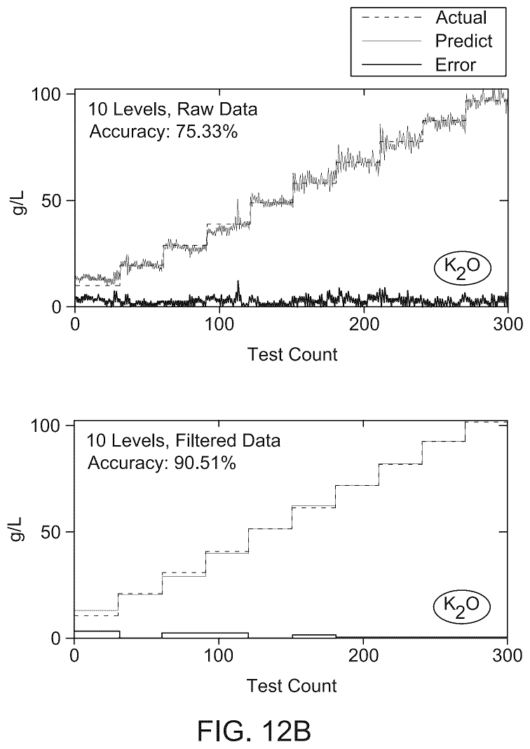

FIG. 11 shows the measured impedance corresponding to 10 levels of NH₃ and K₂O at 10 g/L resolution with sweeping frequencies ranging from 10 kHz to 100 kHz, and the 10-level impedance profiles are also visually distinguishable. FIG. 12 illustrates that processing with the filtered data gives us higher accuracy compared to processing with the raw data: achieving the accuracy of 91.08% for 10 levels of concentration of NH₃ (from 84.34% with the non-filtered NH₃ data) and the accuracy of 90.51% for 10 levels of concentration of K₂O (from 75.33% with the non-filtered K₂O data). This confirms the feasibility and reliability of the developed sensors and sensing circuit.

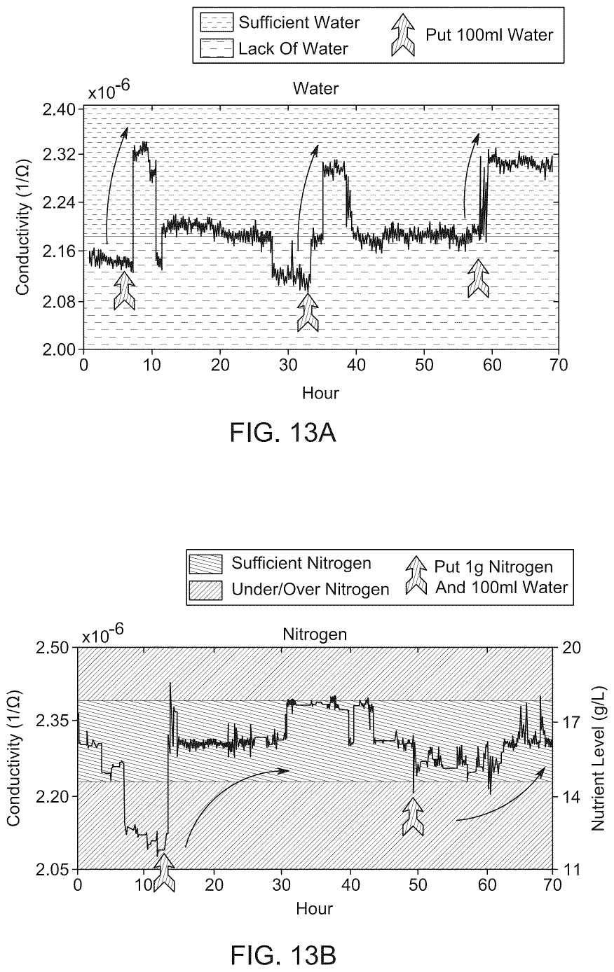

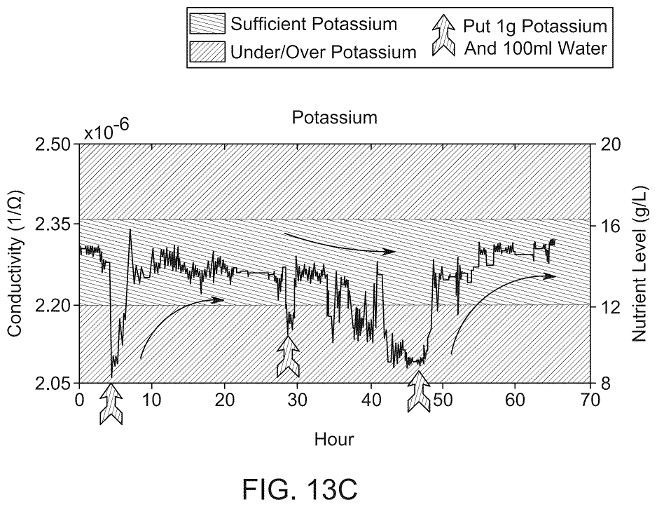

In Vivo Experiments. The biocompatible sensing system is deployed on Burkwood Viburnum and White Bird trees. 100 ml water is first into the soil and observe the measured impedance in the tree body over time. The measured impedance can be shown in FIG. 13(a). As can be seen in the Figure, the tree reaction to the input water can be clearly captured in real-time.

In another experiment, 10 g/L of NH₃ is input into the tree's soil and observe the tree's reaction over time. It is noted that here the tree was not fertilized two weeks before the experiment. The tree's behaviors can be observed as in FIG. 13 (b). The conductivity increase after the tree is fertilized. This indicates that the sensor is sensitive to the nutrient levels change inside the tree body. At the 50th hour, when the conductivity reduces, another 10 g/L of NH₃ is put into the soil. The conductivity then increases to the normal range from 2.22×10−6Ω−1 to 2.37×10−6 Ω−1 corresponding from 14 g/L to 18 g/L. Then the same procedure is followed with K₂O nutrient, and similar reactions from the tree are observed as illustrated in FIG. 13(c). The normal range from 2.20×10−6Ω−1 to 2.32×10−6Ω−1 corresponding from 12 g/L to 15 g/L. A fan is used to generate artificial wind for these experiments. These confirm the feasibility and reliability of the developed sensors and sensing system.

### On the Farm Experiments.

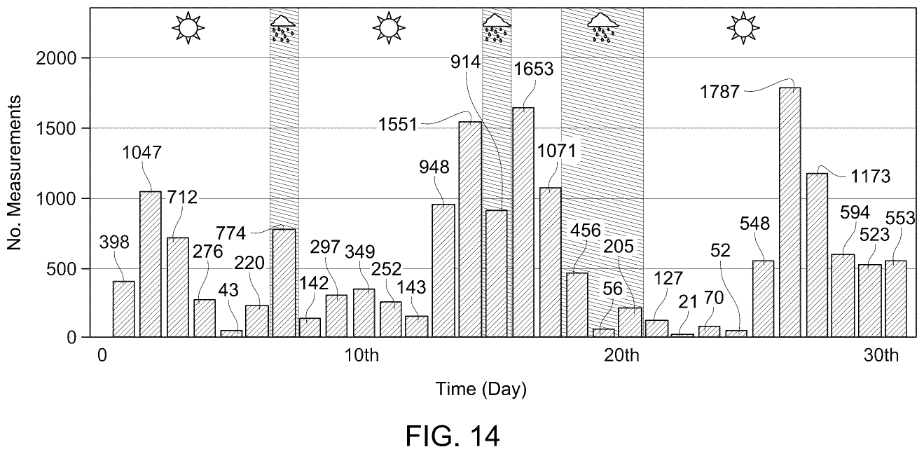

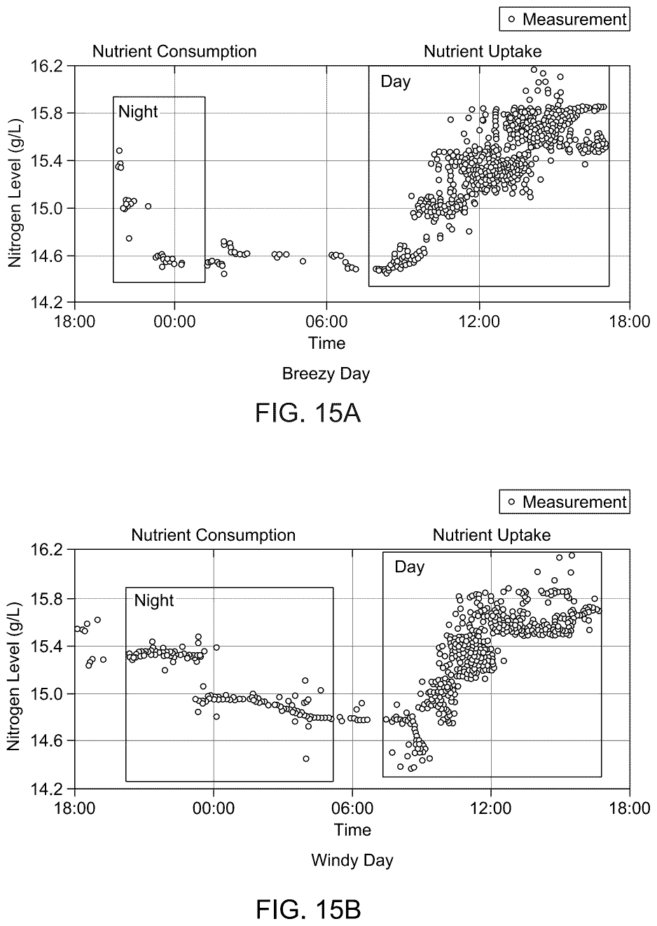

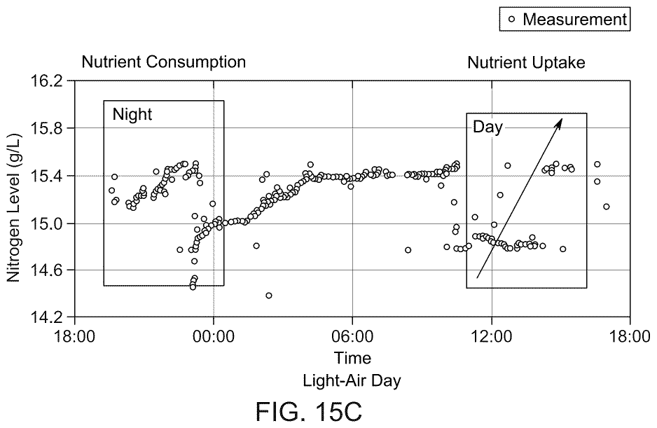

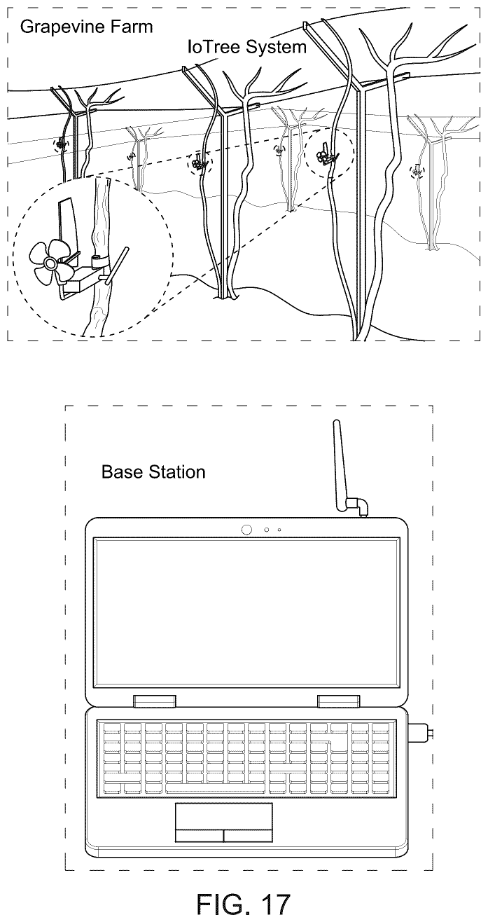

Deployment includes two components: biocompatible sensing system prototypes and a base station, as shown in FIG. 17. Prototypes are attached on grapevine trees farm, while a base station, including a laptop interfacing with the LoRa receiver, is located at a distance of 0.8 km from the farm. FIG. 14 shows that measurements can be obtained from the sensors every day. The number of measures varies each day depending on the weather condition. The total number of sensed measurements ranges from 21 to 1787, which is sufficient for our application. In particular, sensing data as shown in FIG. 15 illustrate that grapevines uptake nutrients during the day and consume nutrients at night. These measured data are matched with nutrient uptake behaviors described in literature. Specifically, when grapevines uptake nutrients, the conductivity increases from 2.23×10⁻⁶Ω⁻¹ to 2.27×10⁻⁶Ω⁻¹, corresponding from 14.4 g/L to 16 g/L for Nitrogen. According to prior work, the levels of Nitrogen fluctuate between normal range.

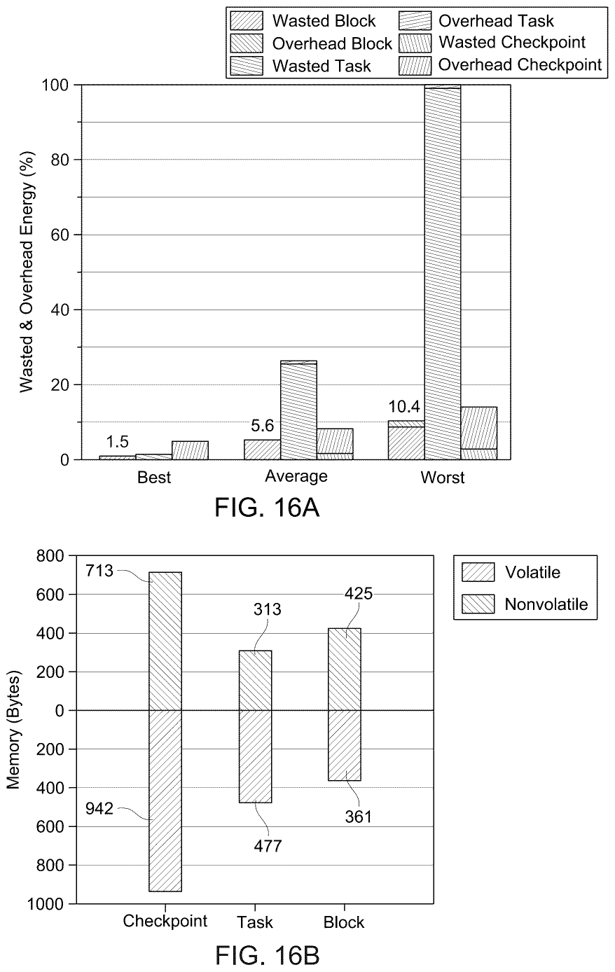

Computing Performance. A block-based approach is compared with checkpoint and tasked-based approaches in terms of energy utilization, computation overhead, and memory utilization on the same hardware platform. Regarding energy utilization, the block-based approach has similar wasted energy to the checkpoint approach but less wasted energy than the block-based approach. If the buffer energy is less than a task's required energy, the task-based approach likely wastes all power in the buffer energy, as shown in FIG. 16 (a). In the average case, our approach outperforms the task-based approach up to 4×. Our approach has similar performance to the task-based approach in terms of overhead while 5× improved compared to the checkpoint-based approach. As for memory utilization, our approach requires less memory than checkpoint up to 2× and has a similar memory footprint to the task-based approach as shown in FIG. 16B.

System Reliability. The biocompatible sensing system is deployed on grapevine trees farm for 30 days with an average number of measurements is 558. The prototypes perform reliably under multiple weather conditions, including windy, rainy, and sunny as illustrated in FIG. 14.

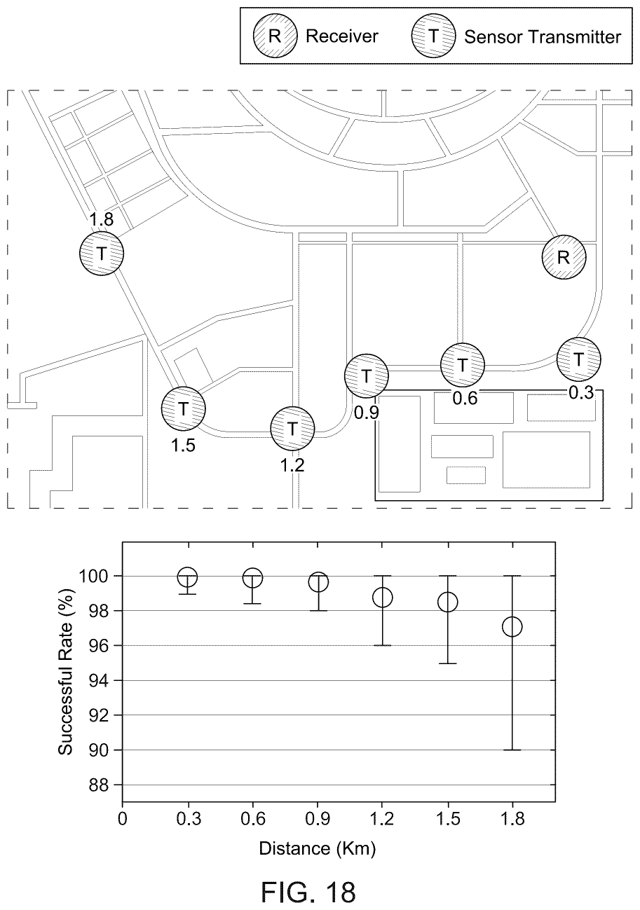

Communication Performance. The performance and reliability of the biocompatible sensing system's long-range communication on the field is evaluated. The base station is placed at a fixed position, while the biocompatible sensing system wearable device is moved to different locations ranging from 0.3 km to 1.8 km from the base station. The system continuously sends data sequences towards the base station. As can be seen from FIG. 18, the communication works reliably up to 1.8 km.

The sensing system can be further optimized to make it more practical for long-term, full-season deployment on various trees at multiple geographical areas. In particular, ion-selective and biocompatible sensing arrays can be implemented to allow us to measure multiple nutrients simultaneously. Ion-selective membranes and Organic Electrochemical Transistor (OECT) are two approaches. The system may also be compatible with more types of trees (e.g., corn, sunflower, rice, and others). The system can be deployed on the smaller body size tree. Multiple geographical locations can be used to confirm the sensitivity and usability of the present technology. The block-based computing approach can be further optimized and applied to other wind-based battery-free systems. While tree soil relationship has been explored in the past, this complex relationship by analyzed further with the data collected from the tree and soil simultaneously utilizing the technology described herein.

The present technology may be embodied as, among other things, a system, method, or computer-product. Accordingly, embodiments may take the form of a hardware embodiment, a software embodiment, or an embodiment combining software and hardware. In one embodiment, the present invention takes the form of a computer program product that includes computer usable instructions embodied on one or more computer readable media and executed by one or more processors.

Computer readable media includes both volatile and nonvolatile media, removable and non-removable media, and media readable by a database, a switch, and various other network devices. Network switches, routers, access points, and related components in some instances act as a means of communication within the scope of the technology. By way of example, computer readable media comprise computer storage media and communications media.

Computer storage media or machine readable media can include media implemented in any method or technology for storing and/or transmitting information or data. Examples of such information include computer-usable instructions, data elements, data structures, programs and program modules, and other data representations.

Communications media generally store computer usable or readable instructions, including data structures and program modules in a modulated data signal. A modulated data signal in some instances can be understood to be a propagated signal that has one or more of its characteristics set or changed to encode information in the signal. Communications media include any information-delivery media. By way of example and not limitation, communications media include wired media, such as a wired network or direct-wired connection, and wireless media such as radio, cellular, spread-spectrum, and other wireless media technologies. Combinations of the above are included with the scope of computer readable media and communications media.

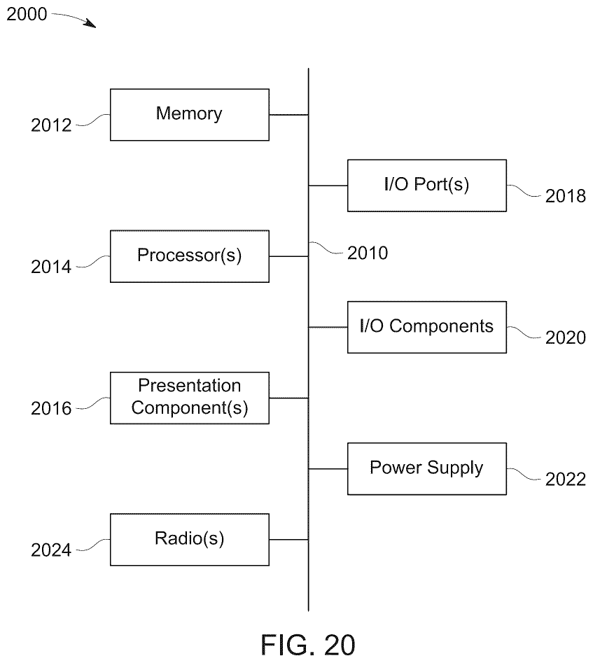

FIG. 20 provides an illustrative operating environment for implementing embodiments of the present invention and designated generally as computing device 2000. Computing device 2000 is merely one example of a suitable computing environment and is not intended to suggest any limitation as to the scope of use or functionality of the invention. Neither should the computing device 2000 be interpreted as having any dependency or requirement relating to any one or combination of components illustrated.

Embodiments of the invention can be described in the general context of computer code or machine-usable instructions, including computer-executable instructions such as program modules, being executed by a computer or other machine (virtual or otherwise), such as a smartphone or other handheld device. Generally, program modules, or engines, including routines, programs, objects, components, data structures etc., refer to code that perform particular tasks or implement particular abstract data types. Embodiments of the invention can be practiced in a variety of system configurations, including hand-held devices, consumer electronics, general-purpose computers, more specialized computing devices, etc. Embodiments of the invention can also be practiced in distributed computing environments where tasks are performed by remote processing devices that are linked through a communications network.

With reference to FIG. 20, computing device 2000 includes a bus 2010 that directly or indirectly couples the following devices: memory 2012, one or more processors 2014, one or more presentation components 2016, input/output ports 2018, input/output components 2020, and an illustrative optional power supply 2022. In some embodiments, devices described herein utilize wired and rechargeable batteries and power supplies. Bus 2010 represents what can be one or more buses (such as an address bus, data bus or combination thereof). Although the various blocks of FIG. 20 are shown with clearly delineated lines for the sake of clarity, in reality, such delineations are not so clear and these lines can overlap. For example, one can consider a presentation component such as a display device to be an I/O component as well. Also, processors generally have memory in the form of cache. It is recognized that such is the nature of the art, and reiterate that the diagram of FIG. 20 is merely illustrative of an example computing device that can be used in connection with one or more embodiments of the present disclosure. Distinction is not made between such categories as “base station” “workstation,” “server,” “laptop,” “hand-held device,” etc., as all are contemplated within the scope of FIG. 7 and reference to “computing device” or “user device”.

Computing device 2000 typically includes a variety of computer-readable media. Computer-readable media can be any available media that can be accessed by computing device 2000, and includes both volatile and non-volatile media, removable and non-removable media. By way of example, and not limitation, computer-readable media can comprise computer storage media and communication media.

Computer storage media include volatile and non-volatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 2000. Computer storage media excludes signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner at to encode information in the signal. By way of example, and not limitation, communication media includes wired media such as a wired network or direct-wired connection, and wireless media such as acoustic, RF, infrared, NFC, Bluetooth, cellular, and other wireless media. Combinations of any of the above should also be included within the scope of computer-readable media.

Memory 2012 includes computer storage media in the form of volatile and/or non-volatile memory. As depicted, memory 2012 includes instructions 2024, when executed by processor(s) 2014 are configured to cause the computing device to perform any of the operations described herein, in reference to the above discussed figures, or to implement any program modules described herein. The memory can be removable, non-removable, or a combination thereof. Illustrative hardware devices include solid-state memory, hard drives, optical-disc drives, etc. Computing device 2000 includes one or more processors that read data from various entities such as memory 2012 or I/O components 2020. Presentation component(s) 2016 present data indications to a user or other device. Illustrative presentation components include a display device, speaker, printing component, vibrating component, etc.

I/O ports 2018 allow computing device 2000 to be logically coupled to other devices including I/O components 2020, some of which can be built in. Illustrative components include a microphone, joystick, touch screen, presentation component, satellite dish, scanner, printer, wireless device, battery, etc.

Many different arrangements of the various components and/or steps depicted and described, as well as those not shown, are possible without departing from the scope of the claims below. Embodiments of the present technology have been described with the intent to be illustrative rather than restrictive. Alternative embodiments will become apparent from reference to this disclosure. Alternative means of implementing the aforementioned can be completed without departing from the scope of the claims below. Certain features and subcombinations are of utility and can be employed without reference to other features and subcombinations and are contemplated within the scope of the claims.

1. A system for monitoring nutrient levels in the body of a tree, the system comprising:
a sensing component comprising a sensor implanted into the tissue of the tree to generate a dataset that corresponds to information about the tree;
a communication component to transmit data, the data comprising at least the generated dataset; and
a base station to receive the data.

2. The system of claim 1, further comprising an energy harvesting component to obtain energy from one or more natural resources and transfer power to at least a portion of the system.

3. The system of claim 1, wherein the sensor comprises one or more biocompatible fiber electrodes.

4. The system of claim 1, wherein the sensing component generates one or more frequency signals.

5. The system of claim 1, wherein the sensing component generates at least one of an impedance or impedance profile for the tree based on the generated dataset.

6. The system of claim 5, wherein a water and/or nutrient level corresponding to the tree is determined from the impedance or impedance profile.

7. The system of claim 1, wherein the sensor is implanted in the xylem of the tree.

8. The system of claim 5, wherein the impact of environmental noise is reduced or minimized.

9. The system of claim 2, further comprising a natural resource indicator component to optimize the obtained energy.

10. The system of claim 1, wherein at least a portion of the components operate in response to a block-based intermittent computing model.

11. A computer-implemented method for monitoring nutrient levels in the body of a tree, the method comprising:
implanting one or more sensors into a tree body;
capturing one or more signals from the tree to generate a dataset corresponding to the tree;
determining an impedance and/or impedance profile for the tree; and
transmitting the at least one of the dataset, the impedance and/or impedance profile to a base station.

12. The method of claim 11, further comprising compressing the at least one of the dataset, the impedance and/or impedance profile prior to the transmitting.

13. The method of claim 11, further comprising generating one or more frequency signals.

14. The method of claim 11, wherein the transmitting is carried out continuously.

15. The method of claim 11, further comprising processing the signals.

16. The method of claim 11, further comprising harvesting energy from one or more natural resources.

17. A computer storage medium storing computer-useable instructions that, when used by one or more computing devices, cause the one or more computing devices to perform operations comprising:
capturing one or more signals from the tree to generate a dataset corresponding to the tree;
determining an impedance and/or impedance profile for the tree; and
transmitting the at least one of the dataset, the impedance and/or impedance profile to a base station.

18. The computer storage medium of claim 17, further comprising harvesting energy from one or more natural resources.

19. The computer storage medium of claim 17, further comprising generating one or more frequency signals.

20. The computer storage medium of claim 17, further comprising processing the captured signals.
